(19) United States
(12) Plant Patent Application Publication
(10) Pub. No.: US 2008/0263734 P1
(43) Pub. Date: Oct. 23, 2008

(54) Calla lily plant named 'Hot Cherry BLZ'

(76) Inventors: Andrew G.F. Warren, Tauranga (NZ); Jacobus P. Spaans, Tauranga (NZ)

Correspondence Address: JONDLE & ASSOCIATES P.C., 858 HAPPY CANYON ROAD SUITE 230, CASTLE ROCK, CO, 80108, US

(21) Appl. No.: 11/787,815

(22) Filed: Apr. 18, 2007

(51) Int. Cl. A01H 5/00

(52) U.S. Cl. PLT/313

(57) ABSTRACT

A calla lily plant particularly distinguished by a purple-red spathe, low density maculation of the leaves that is visible from both sides and which produces 2 to 4 inflorescences per 3-cm tuber, is described.

### GENUS AND SPECIES

Zantedeschia spp.

### VARIETY DENOMINATION

‘Hot Cherry BLZ’

### BACKGROUND OF THE NEW PLANT

The present invention comprises a new and distinct cultivar of calla lily, botanically known as Zantedeschia spp., and hereinafter referred to by the cultivar name ‘Hot Cherry BLZ’. The new cultivar was asexually reproduced from micropropagation resulting from the cross of the female parent calla lily, an unnamed seedling of Zantedeschia spp. (unpatented) with a pink spathe and the male parent calla lily ‘Majestic Red’ (patent status unknown) with a red/burgundy spathe.

The new cultivar was created in Tauranga, New Zealand and has been asexually reproduced repeatedly by micropropagation in India over a two-year period. The present invention has been found to retain its distinctive characteristics through successive propagations.

Plant Breeder's Rights for this cultivar were applied for in New Zealand on Oct. 6, 2006.

### SUMMARY OF THE INVENTION

The following are the most outstanding and distinguishing characteristics of this new cultivar when grown under normal horticultural practices in Tauranga, New Zealand.

1. A purple-red spathe;

2. Low density maculation of the leaves that is visible from both sides; and

3. Produces 2 to 4 inflorescences per 3-cm tuber.

### DESCRIPTION OF THE PHOTOGRAPHS

This new calla lily plant is illustrated by the accompanying photographs which show the mature inflorescence and foliage of the plant. The colors shown are as true as can be reasonably obtained by conventional photographic procedures.

FIG. 1 shows a close-up of the mature inflorescence.

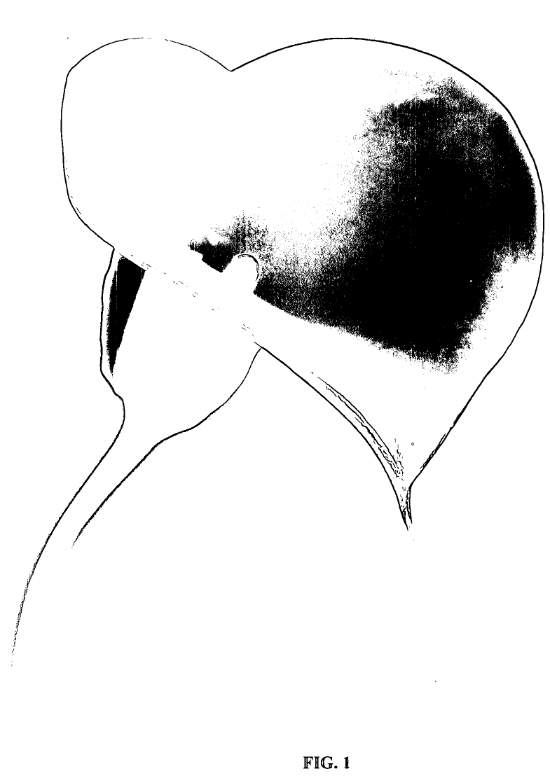

FIG. 2 shows a close of a mature leaf with maculation.

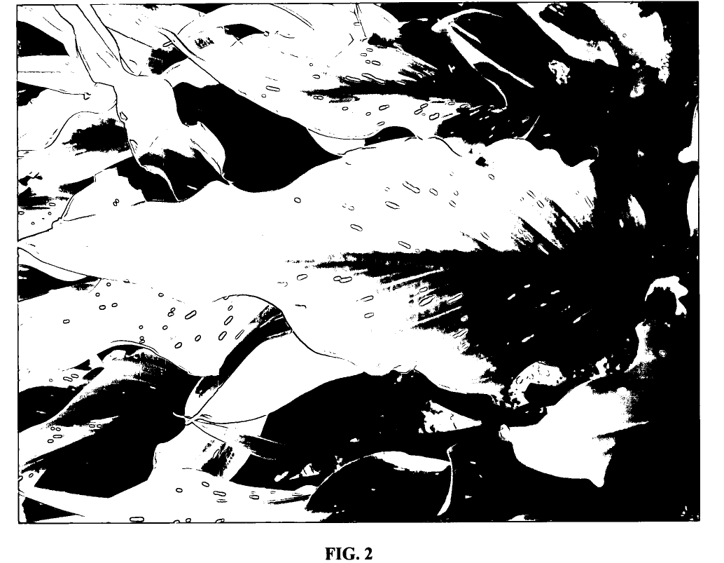

### DESCRIPTION OF THE NEW CULTIVAR

The following detailed description sets forth the distinctive characteristics of ‘Hot Cherry BLZ’. The data that define these characteristics were collected from asexual reproductions carried out in Tauranga, New Zealand. The plant history was taken on two-year-old plants based on a 3-cm diameter tuber under New Zealand summer conditions. Color references are primarily to the RHS Colour Chart of The Royal Horticultural Society of London (RHS).

### DETAILED BOTANICAL DESCRIPTION

Classification:
   Family.—Araceae.
   Botanical name.—Zantedeschia spp.
   Common name.—Calla lily.
Plant:
   Form.—Erect.
   Height of the leaf canopy above the soil.—45 cm to 55 cm.
   Height of top of inflorescence above the soil.—50 cm to 60 cm.
   Diameter.—25 cm to 35 cm.
   Number of inflorescences per tuber size.—Tuber diameter 3 cm: 2 to 4 Tuber diameter 4 cm: 3 to 6 Tuber diameter 5 cm: 6+.
   Time to produce a finished flowering plant.—6 to 10 weeks depending on the climate and environment.
   Lastingness of inflorescence on the plant.—2 to 3 weeks.
Growing points:
   Average number per plant.—2 to 4.
   Character.—Strong.
   Color.—Upper surface: RHS 137C Lower surface: RHS 146D Base: RHS 155D.
Leaves:
   Size.—Width: 8 cm to 11 cm Length: 22 cm to 28 cm.
   Shape.—Ovate.
   Color.—Upper surface: RHS 137B Lower surface: RHS 137D.
   Maculation (leaf spots).—General: Maculation is only on the upper surface of the leaf but is visible from the lower surface Density: Low density Color: RHS 155C (white).
   Apex.—Acute to apiculate.
   Base.—Attenuate to shortly attenuate.
   Margin.—Entire, moderately wavy.
   Margin color.—Green.
   Venation pattern.—Pinnate.
   Surface texture.—Leathery.
   Petiole.—Average number per tuber: 8 to 12 Length: 15 cm to 25 cm Diameter: 0.6 cm to 1.2 cm Color: Upper portion: RHS 137C Base: RHS 146D Texture: Smooth Character: Strong Sheath: Width: 0.6 cm to 0.8 cm Length: 2 cm to 5 cm Color: RHS 137C (green).
Roots:
   Color.—RHS 155D (white)
   Branching.—Not branched, attached to the top of the tuber at apices.
Inflorescence:
   Spathe (flower body).—Size: Length: 7 cm to 9 cm Height: 8 cm to 10 cm Width: 5 cm to 6 cm Color: Upper part: RHS 60A Lower part: RHS 60C Veins: RHS 59A Shape: Fluted Texture: Smooth Apex: Caudate Base: Attenuate Margin: Entire
   Fragrance.—Absent.
   Spadix (reproductive organs).—Size: Length: 2.5 cm to 3.5 cm Diameter: 0.6 cm to 0.8 cm Color: Base: RHS 60B Upper part: RHS 15B Position relative to spathe: Upright Shape: Elongated Apex: Rounded.
   Peduncle (flower stem).—Length: 35 cm to 55 cm Diameter: 0.7 cm to 1.1 cm Color: Base: RHS 155A Upper part: RHS 60B Texture: Smooth Strength: Strong.
Reproductive organs:
   Location of female organs.—Basal position of the spadix.
   Location of male organs.—Upper position of the spadix.
   Perianth (the floral envelope, calyx and corolla).—Conspicuous.
   Stamens.—Not visible before pollen release.
   Pollen color.—RHS 155D (white).
   Pistil.—Number: 25 to 35 per spadix Color: RHS 17B.
Fruit:
   General.—Whole fruit is 2.5 cm to 3.5 cm wide and 4.0 cm to 5.0 cm long; individual berries are round and 0.5 cm to 0.7 cm in circumference.
   Number.—8 to 15 berries per plant.
   Color.—RHS 144A.
Disease and insect resistance:
   Disease resistance.—No susceptibility noted to date.
   Pest resistance.—No susceptibility noted to date.

### COMPARISON WITH PARENTAL AND COMMERCIAL CULTIVARS

When the instant plant is compared to the female parent, an unnamed seedling of Zantedeschia spp. (unpatented), ‘Hot Cherry BLZ’ has darker pink flowers and larger flowers than the female parent.

When ‘Hot Cherry BLZ’ is compared to the male parent, ‘Majestic Red’ (patent status unknown), ‘Hot Cherry BLZ’ has a brighter (lighter) flower color and more inflorescences than the male parent.

‘Hot Cherry BLZ’ differs from the commercial cultivar ‘Hot Lips’ (U.S. Plant Pat. No. 15,260) in that ‘Hot Cherry BLZ’ has a higher flower count, more consistently shaped spathes and the spathe color is more purple-red, while the spathe color of ‘Hot Lips’ is more pink.

I claim:

1. A new and distinct cultivar of calla lily plant as shown and described herein.
